(19) United States
(12) Patent Application Publication
(10) Pub. No.: US 2004/0176745 A1
(43) Pub. Date: Sep. 9, 2004

(54) Wound drain with elongated intermediate section of enhanced radial strength

(76) Inventor: Scott Ferguson, Atlanta, GA (US)

Correspondence Address: JOHN S. PRATT, ESQ, KILPATRICK STOCKTON, LLP, 1100 PEACHTREE STREET, SUITE 2800, ATLANTA, GA, 30309, US

(21) Appl. No.: 10/384,191

(22) Filed: Mar. 7, 2003

(51) Int. Cl. A61M 27/00

(52) U.S. Cl. 604/543

(57) ABSTRACT

A wound drain comprises an elongated drain portion, a suction tubing, and a connector. The drain portion includes a plurality of longitudinal lumens, and the exterior wall of the drain portion includes slots, holes, or the like for placing at least one of the longitudinal lumens in communication with the ambient surrounding the drain portion. The connector connects a first end of the drain portion to a first end of the suction tubing such that at least one of the longitudinal lumens of the drain portion is placed in fluid communication with the lumen of the suction tubing. The connector exhibits increased radial strength in comparison to the drain portion and the suction tubing. In one embodiment the connector is approximately one-third the length of the elongated drain portion.

In another aspect, the wound drain of the present invention presents a smooth profile from the suction tubing to the connector, and along the length of the connector, eliminating any edges or step ups that can catch on and tear at tissue when the wound drain is removed from the patient.

### TECHNICAL FIELD

The present invention relates generally to wound drains and relates more specifically to a wound drain with an elongated intermediate section of enhanced radial strength.

### BACKGROUND OF THE INVENTION

Wounds, whether they be from the result of injuries or surgical interventions, typically exude fluid as part of the healing process. Where the wound is beneath the skin, it is beneficial to provide a drainage device to facilitate drainage of the exudate from the wound to a location outside the body.

A typical wound drain is disclosed in U.S. Pat. No. 4,398,910 to Blake. A drain portion has radial walls to divide the central core into a plurality of longitudinal lumens. Elongated slots in the outer walls provide entry from the surrounding tissue into each longitudinal lumen. One end of the drain portion is connected to a tubing that is hooked up to a suction source. Thus each longitudinal lumen of the drain portion serves to draw exudate through the longitudinal slots and into the lumen, whence it is drawn into the tubing and out of the body. If a clot or other blockage occludes a portion of a longitudinal slot, exudate is simply drawn into the lumen on either side of the blockage, and the blockage does not interfere with normal operation of the device.

The aforementioned U.S. Pat. No. 4,398,910 discloses drains that are fully fluted and drains that are three-quarters fluted. In the fully fluted drains, the drain section contains longitudinal slots all the way up to the point that the drain section connects to the suction tubing. In the three-quarter fluted embodiment, three-fourths of the drain section contains longitudinal slots, and the remaining one-quarter of the drain section is a “transition” section adapted for passage through non-exuding tissue. This transition section is made from the same soft material as the remaining three-quarters of the drain section and is thus easily compressed, lending itself to be easily radially compressed by healthy tissue. In addition, the transition section bends easily and tends to collapse under the internal suction when so bent. Also, the transition section of the drain disclosed in the aforementioned U.S. Pat. No. 4,398,910 contains multiple (four) small lumens, which are prone to clogging.

Thus there is a need for a wound drain that is less than fully fluted that has a transition section that is resistant to radial compression, is not easily compressed by healthy tissue, and does not bend easy and thus does not tend to collapse under internal suction pressures when so bent.

### SUMMARY OF THE INVENTION

Stated generally, the present invention comprises a wound drain including an elongated drain portion, a suction tubing, and a connector. The drain portion includes a plurality of longitudinal lumens, and the exterior wall of the drain portion includes slots, holes, or the like for placing the longitudinal lumens in communication with the ambient surrounding the drain portion. The connector connects a first end of the drain portion to a first end of the suction tubing such that the longitudinal lumens of the drain portion are placed in fluid communication with the lumen of the suction tubing. The connector exhibits increased radial strength in comparison to the drain portion and the suction tubing. In one embodiment the connector is approximately one-third the length of the elongated drain portion.

In another aspect, the wound drain of the present invention presents a smooth profile from the suction tubing to the connector, and along the length of the connector, eliminating any edges or step ups that can catch on and tear at tissue when the wound drain is removed from the patient.

Thus it is an object of the present invention to provide a connector for traversing non-exuding tissue that exhibits increased radial strength in comparison to the drain portion and the suction tubing.

It is another object of the present invention to provide a wound drain that presents a smooth profile from the suction tubing to the connector, and along the length of the connector, eliminating any edges or step ups that can catch on and tear at tissue.

It is still another object to provide a wound drain that minimizes the possibility of clogging occurring at the transition between the drain portion and the suction tubing.

Other objects, features, and advantages of the present invention will become apparent upon reading the following specification, when taken in conjunction with the drawings and the appended claims.

### BRIEF DESCRIPTION OF THE DRAWINGS

FIG. 1 is an orthogonal view of a drain section of a wound drain according to a disclosed embodiment of the invention.

FIG. 2 is an end view of the drain section of FIG. 1.

FIG. 3 is an orthogonal view of a suction tubing of a wound drain according to a disclosed embodiment of the invention.

FIG. 4 is an end view of the suction tubing of FIG. 3.

FIG. 5 is a side view of a first embodiment of a connector of a three-quarter fluted wound drain according to a disclosed embodiment of the invention.

FIG. 6 is a cutaway view taken along line 6-6 of FIG. 5.

FIG. 7 is a front view of the connector of FIG. 5.

FIG. 8 is a rear view of the connector of FIG. 5.

FIG. 9 is an orthogonal view of the connector of FIG. 5.

FIG. 10 is an exploded orthogonal view showing the assembly of the drain section of FIG. 1 and the suction tubing of FIG. 3 onto the connector of FIG. 5.

FIG. 11 is a partial cutaway view showing the drain section of FIG. 1 and the suction tubing of FIG. 3 coupled to the connector of FIG. 5 to form a wound drain.

FIG. 12 is a schematic diagram showing the placement of the wound drain of FIG. 11 with the drain section positioned within a wound and the suction tubing connected to a pump.

FIG. 13 is an orthogonal view of an alternate embodiment of a drain section of a disclosed wound drain.

FIG. 14 is an end view of the drain section of FIG. 13.

FIG. 15 is a top view of a connector for use with the drain section of FIG. 13 to form a three-quarter fluted wound drain.

FIG. 16 is a side view of the connector of FIG. 15.

FIG. 17 is a cross-sectional view taken along line 17-17 of FIG. 16.

FIG. 18 is a cross-sectional view taken along line 18-18 of FIG. 15.

FIG. 19 is a front view of the connector of FIG. 15.

FIG. 20 is a rear view of the connector of FIG. 15.

FIG. 21 is an exploded orthogonal view showing the assembly of the drain section of FIG. 13 and the suction tubing of FIG. 3 onto the connector of FIG. 15.

FIG. 22 is a partial top cutaway view showing the drain section of FIG. 13 and the suction tubing of FIG. 3 coupled to the connector of FIG. 15 to form a wound drain.

FIG. 23 is a partial side cutaway view showing the drain section of FIG. 13 and the suction tubing of FIG. 3 coupled to the connector of FIG. 15 to form a wound drain.

FIG. 24 is a side view of a connector for use with the drain section of FIG. 1 and the suction tubing of FIG. 3 to form a full-fluted wound drain.

FIG. 25 is a section view taken along line 25-25 of FIG. 24.

FIG. 26 is a front view of the connector of FIG. 24.

FIG. 27 is a rear view of the connector of FIG. 24.

FIG. 28 is an orthogonal view of the connector of FIG. 24.

FIG. 29 is a full-fluted wound drain comprising the connector of FIG. 24, the drain section of FIG. 1 and the suction tubing of FIG. 3.

FIG. 30 is a top view of a connector for use with the drain section of FIG. 13 and the suction tubing of FIG. 3 to form a full-fluted wound drain.

FIG. 31 is a side view of the connector of FIG. 30.

FIG. 32 is a section view taken along line 32-32 of FIG. 31.

FIG. 33 is a section view taken along line 33-33 of FIG. 30.

FIG. 34 is a front view of the connector of FIG. 30.

FIG. 35 is a rear view of the connector of FIG. 30.

FIG. 36 is an orthogonal view of the connector of FIG. 30.

### DETAILED DESCRIPTION OF THE DISCLOSED EMBODIMENT

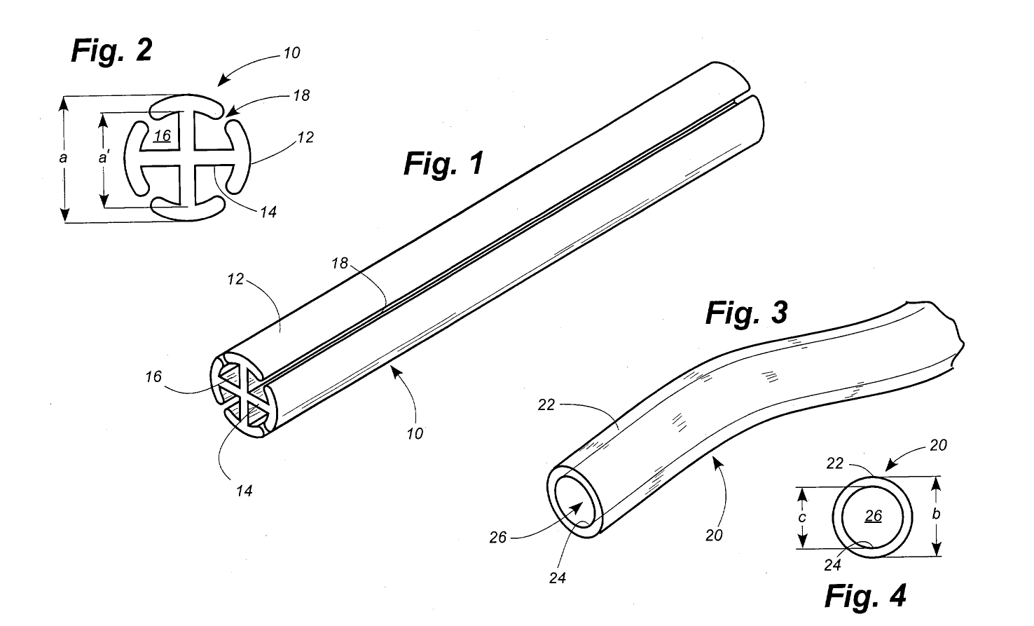

Referring now to the drawings, in which like numerals indicate like elements throughout the several views, FIGS. 1 and 2 illustrate a drain section 10 of a wound drain of the present invention. The drain section 10 comprises an elongated, generally tubular outer wall member 12 and a plurality of radial interior wall members 14. The radial walls 14 divide the drain section into a plurality of longitudinal lumens 16. Each longitudinal lumen communicates with the ambient by way of a longitudinal slot 18 formed in the outer wall 12. The drain section 10 has an outer diameter a and an inner diameter a′.

Referring now to FIGS. 3 and 4, the wound drain of the present invention further comprises a suction tubing 20. The suction tubing 20 is an elongated tubing comprising an outer wall 22 and an inner wall 24. The tubing 20 defines a longitudinal lumen 26. The tubing 20 has an outer diameter b and an inner diameter c.

All that remains of the wound drain is a connector for connecting the drain section 10 to the suction tubing 20. The drain section, with its series of elongated grooves and channels, is said to be “fluted.” By convention, a drain section is typically “full fluted” or “three-quarters fluted.” That is, the entire section that is attached to the suction tubing 20 is fluted, or only three-quarters of the section that is attached to the suction tubing 20 is fluted. The three-quarters fluted wound drain is intended for use on wounds lying beneath the surface of the skin, where the wound drain must traverse non-exuding tissue before exiting the skin.

On the wound drain of the present invention, the non-fluted portion of the three-quarter fluted wound drain is provided entirely by the connector. That is, for a conventional twelve-inch drain, only nine inches of which will be fluted, the remaining three inches is comprised of the connector.

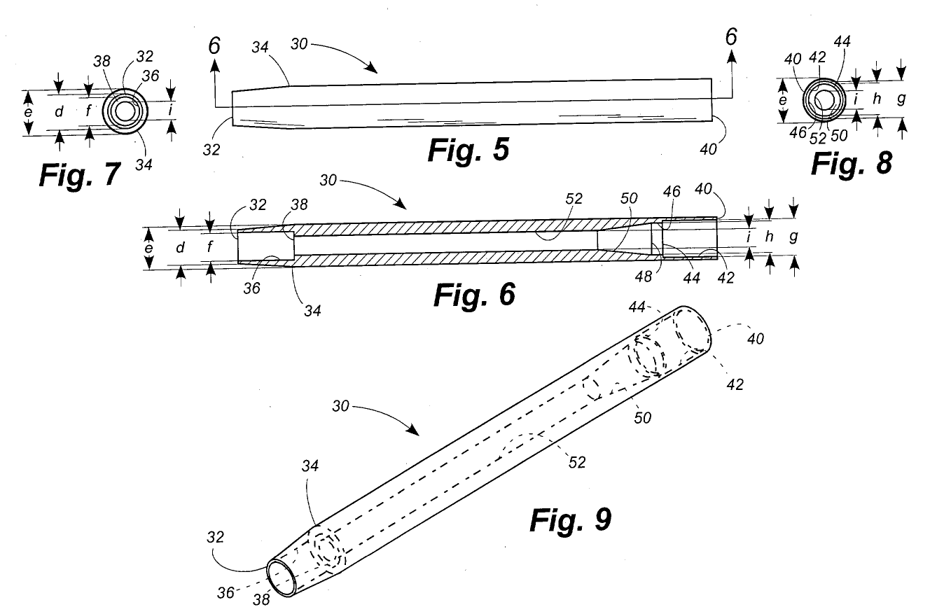

FIGS. 5-9 illustrate a connector 30 for use with the drain section 10 and suction tubing 20 previously described. The connector 30 is designed to adapt the drain section 10 and suction tubing 20 to form a three-quarter fluted wound drain. Toward that end, the connector 30 is approximately three inches long, and the drain section 10 with which the connector is used is marginally over nine inches in length. Stated differently, the connector is approximately one-third the length of the drain section.

Referring now to FIGS. 5-9, the connector 30 has a forward end 32 with an outer diameter d. The diameter of the connector flares outward as the longitudinal location along the connector moves rearward until it reaches a point 34, where the diameter e of the connector remains constant for the remainder of its length.

A bore 36 is formed in the forward end 32 of the connector 30. The bore 36 has a diameter f. The bore 36 has a base 38.

The connector 30 has a rearward end 40 with an outer diameter e. A bore 42 is formed in the rearward end 40 with a diameter g. The bore 42 has a base 44. A counterbore 46 is formed in the base 44 coaxial with the bore 42. The counterbore 46 has a diameter h. At the rear end 48 of the counterbore 46, there is a taper 50 to still another bore 52. The bore 52 has a diameter i and extends a major portion of the length of the connector 30 through the base 38 of the bore 36 at the forward end of the connector.

The connector is comprised of silicone rubber, polyvinyl chloride (PVC), or other suitable polymer such as C-Flex®. This base material may have anywhere from 1% to 40%, preferably 13% ±2%, barium sulfate for radiopacity. Alternatively titanium dioxide or other suitable material may be substituted for radiopacity. The material of the connector has a hardness of from 30 to 80 on the Shore A scale, preferably around 50.

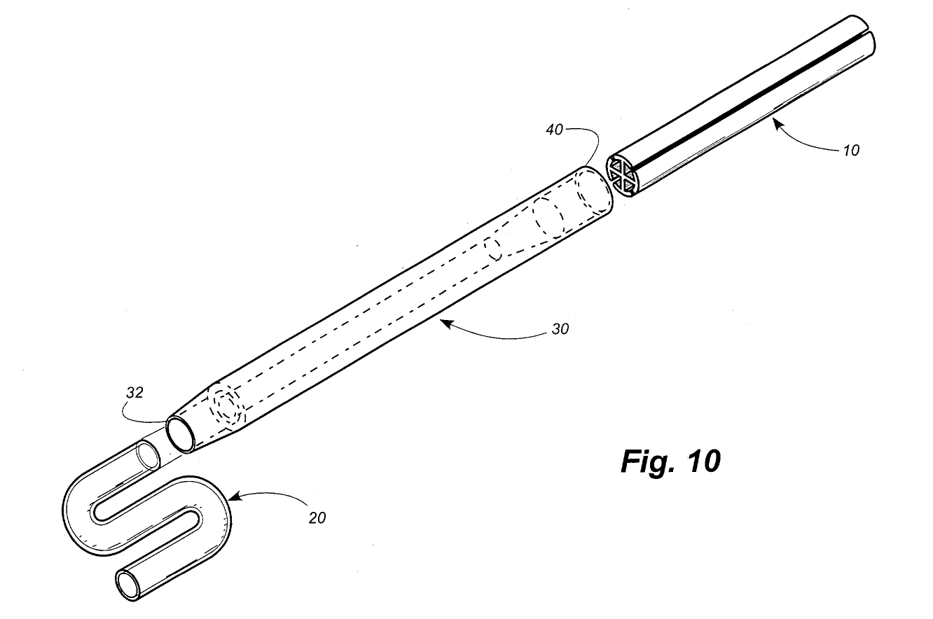

Referring now to FIG. 10, the forward end 32 of the connector 30 is adapted to receive an end of the suction tubing 20. The rearward end of the connector 30 is adapted to receive an end of the drain section 10.

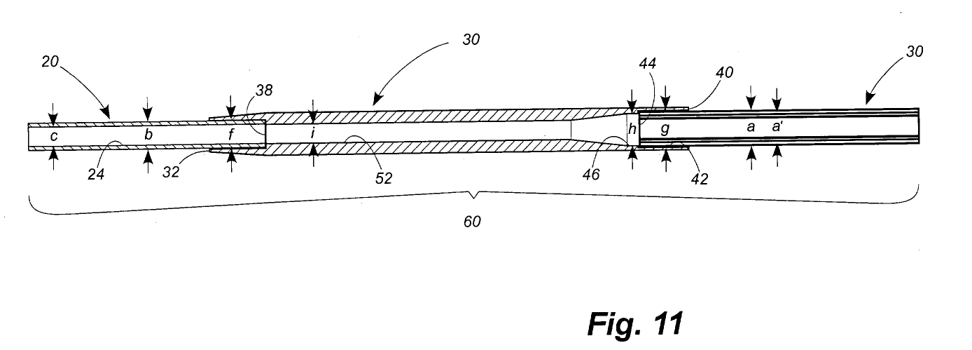

FIG. 11 shows the assembled wound drain 60. The rearward end 40 of the connector 30 is adapted to receive an end of the drain section 10. The drain section 10 is designed to be received within the bore 42, with the end of the drain section 10 abutting the base 44 of the bore 42. The outer diameter a of the drain section 10 is configured to be snugly received within the inner diameter g of the bore 42. The inner diameter a′ of the drain section 10 approximately corresponds to the diameter h of the counterbore 46 of the connector. Thus fluid flowing through the lumen 16 of the drain section 10 will flow smoothly and without interruption from the drain section 10 into the counterbore 46.

Similarly, the forward end 32 of the connector 30 is adapted to receive an end of the suction tubing 20. The outer diameter b of the suction tubing 20 is designed to be received snugly within the inner diameter f of the forward end 32 of the connector 30. Also, the inner diameter c of the suction tubing 20 is designed to correspond to the diameter i of the elongated bore 52 of the connector. Thus when the suction tubing is inserted into the bore 36 in the forward end 32 of the connector 30, the suction lumen formed by the lumen 24 of the suction tubing and the bore 52 of the connector is substantially uninterrupted and of substantially constant diameter.

Both the drain section 10 and the suction tubing 20 are bonded to the connector 30 with silicone adhesive.

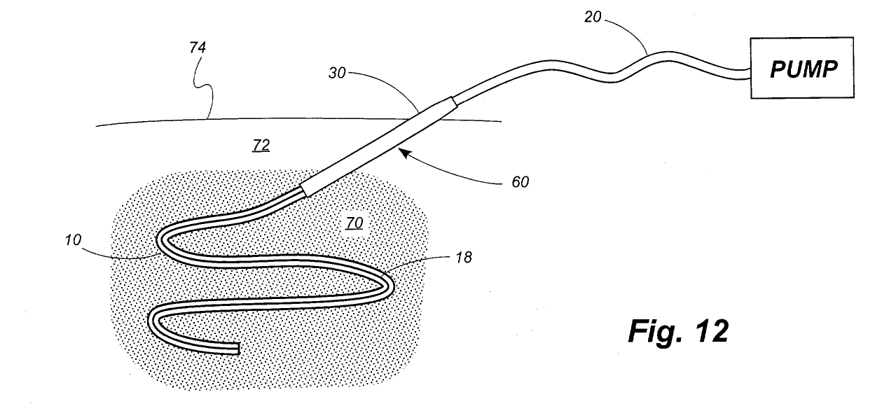

FIG. 12 illustrates the wound drain 60 implanted within the tissues of a patient. The shaded area 70 indicates the wound, and specifically exuding tissues. The unshaded area 72 surrounding the wound indicates healthy, non-exuding tissue, and the line 74 above the healthy tissue indicates the skin. In FIG. 12 the drain section 10 winds through the wound 70. The suction tubing 20, one end of which is connected to a pump 80, communicates a low pressure through the connector 30 and through the lumens 16 of the drain section 10. This low pressure draws exudate through the slots 18 in the drain section walls 12 and into the longitudinal lumens, where it is drawn up the drain section 10, through the connector 30, up the suction tubing 20, and out of the wound drain 60.

The wound drain 60 depicted in FIG. 12 is a three-quarter fluted design. Thus for a twelve-inch wound drain, nine inches of the drain is fluted. To create three inches of non-fluted drain, a three-inch connector 30 is provided. The three-inch connector 30 traverses the healthy tissue 72 between the wound 70 and the skin 74. Thus there is no vacuum trying to draw healthy tissue into the wound drain, clogging the drain and injuring healthy tissue. Further, because the connector 30 exhibits greater radial strength than the suction tubing 20, the connector resists collapse when forces are exerted upon it by the healthy tissue. And because the connector 30 tends to maintain its shape in opposition to forces exerted upon it by surrounding tissue, the lumens of the connector tend to maintain their shape, making the wound drain 60 less likely to clog. In addition, because all of the lumens of the drain section 10 terminate into a single lumen 46 of the connector 30, the wound drain 60 rapidly transitions over a short (0 313″) distance within the non-fluted portion (i.e. the connector 30) from four lumens to a single large lumen, which reduces the likelihood that particulate debris from the exuding wound will clog the wound drain. This design is in contrast to the design in the aforementioned U.S. Pat. No. 4,398,910, which contains four small lumens throughout the non-fluted portion which are prone to clogging due to particulate and debris from the exuding wound.

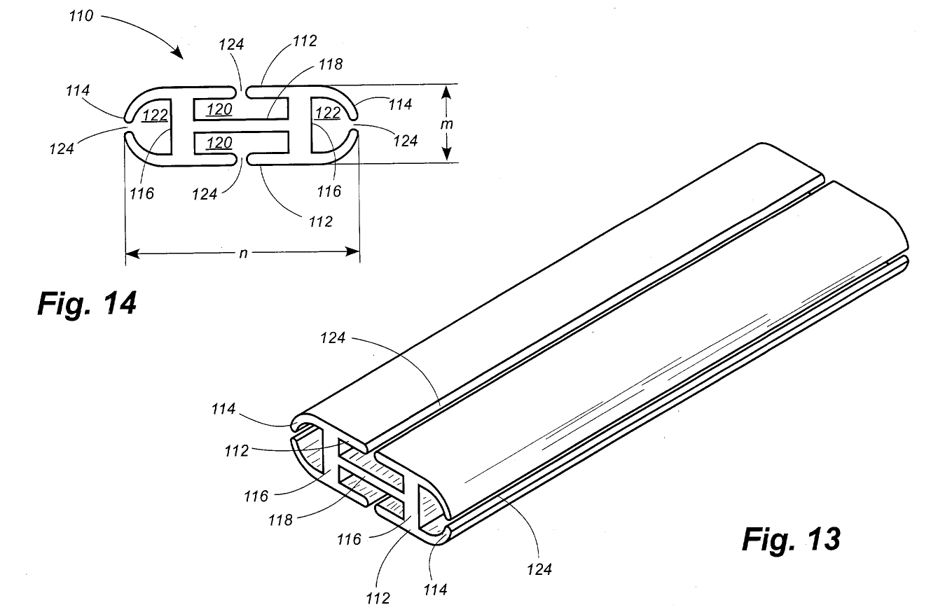

Referring now to FIGS. 13 and 14, an alternate embodiment of a drain section 110 is illustrated. The drain section 110 is a rounded rectangle with flat upper and lower walls 112 and rounded end walls 114. The drain section 110 has a height m and a width n.

Two vertical interior walls 116 connect the upper and lower walls 112. A horizontal interior wall 118 connects the midpoints of the vertical interior walls 116. The vertical interior walls 116 and the horizontal interior wall 118 form an “H” shape supporting the exterior walls 112, 114.

Still referring to FIGS. 13 and 14, elongated channels or lumens 120 are formed between the vertical interior walls 116 above and below the horizontal interior wall 118. Two more elongated channels or lumens 122 are formed on the outsides of the vertical interior walls 116 and inside the rounded end walls 114. Elongated slots 124 are formed in the outer walls 112, 114 to place the elongated channels 120, 122 in communication with the ambient surrounding the drain section 110.

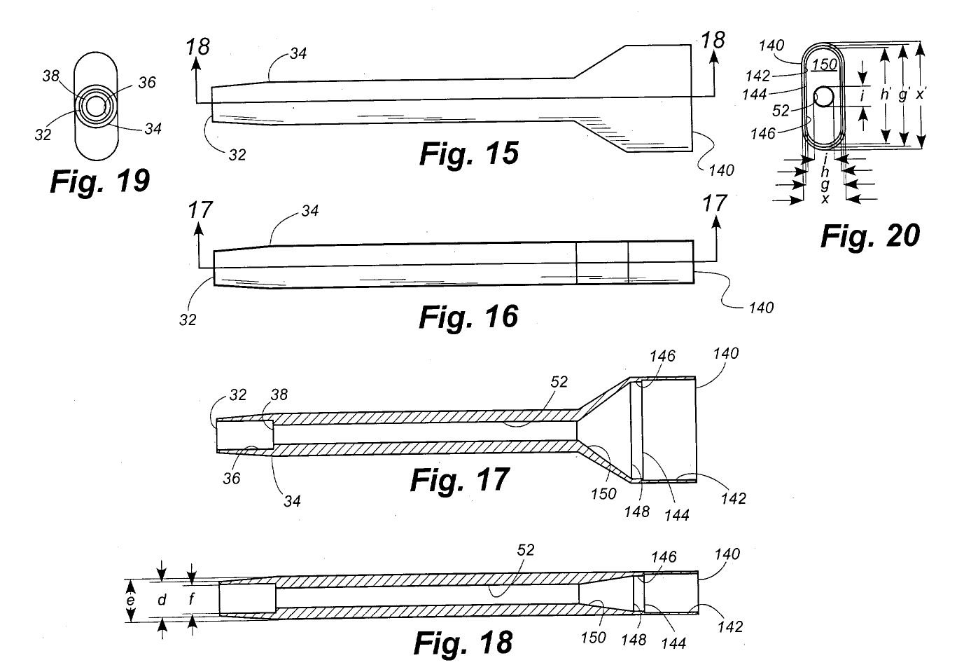

FIGS. 15-20 depict a connector 130 adapted for use with the suction tubing 20 of FIGS. 3 and 4 and the flat drain section 110 of FIGS. 13 and 14. The connector 30 is designed to adapt the drain section 110 and suction tubing 20 to form a three-quarter fluted wound drain. Toward that end, the connector 130 is approximately three inches long, and the drain section 10 with which the connector is used is marginally over nine inches in length. Stated differently, the connector is approximately one-third the length of the drain section.

Referring now to FIGS. 15-20, the connector 130 has a forward end 32 with an outer diameter d. The diameter of the connector flares outward as the longitudinal location along the connector moves rearward until it reaches a point 34, where the diameter e of the connector remains constant for a major portion of its length.

A bore 36 is formed in the forward end 32 of the connector 130. The bore 36 has a diameter f The bore 36 has a base 38.

The connector 30 has a rearward end 140. The rearward end is in the shape of a rounded rectangle having an outer height x and an outer width x′. A rounded rectangular cavity 142 is formed in the rearward end 140 with a height g and a width g′. The rounded rectangular cavity 142 has a base 144. A rounded rectangular cavity 146 is formed in the base 144 coaxial with the rounded rectangular cavity 142. The rounded rectangular cavity 146 has a height h and a width h′. At the rear end 148 of the rounded rectangular cavity 146, there is a taper 150 to a circular bore 52. The circular bore 52 has a diameter i and extends a major portion of the length of the connector 130 through the base 38 of the bore 36 at the forward end of the connector.

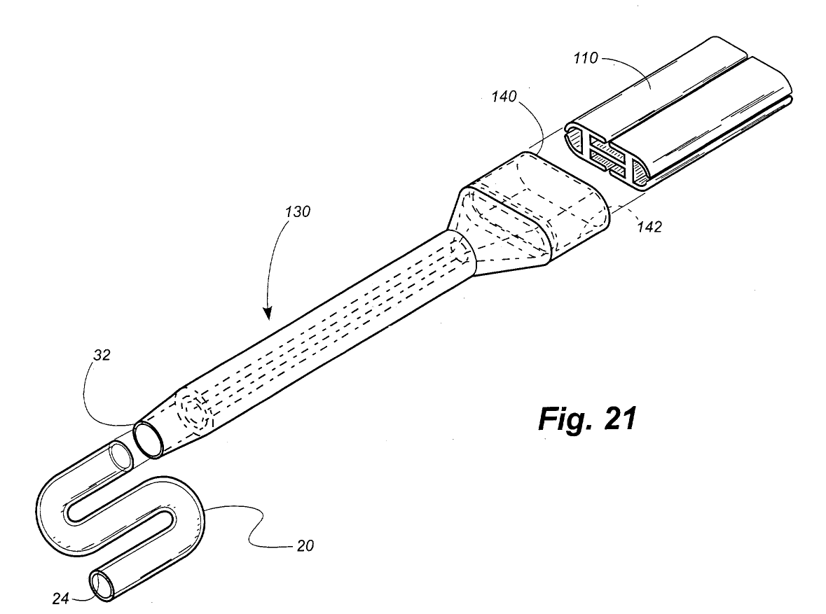

FIG. 21 illustrates how the suction tubing 20 and the flat drain section 110 engage the connector 130. The forward end 32 of the connector 130 is adapted to receive an end of the suction tubing 20. The outer diameter b of the suction tubing 20 is designed to be received snugly within the inner diameter f of the forward end 32 of the connector 130. Also, the inner diameter c of the suction tubing 20 is designed to correspond to the diameter i of the elongated bore 52 of the connector. Thus when the suction tubing is inserted into the bore 36 in the forward end 32 of the connector 130, the suction lumen formed by the lumen 24 of the suction tubing and the bore 52 of the connector is substantially uninterrupted and of substantially constant diameter.

As can also be seen in FIG. 21, the flat drain section 110 is aligned such that its width n (FIG. 14) is aligned with the width x′ (FIG. 20) of the rear portion of the connector 130. The height and width m, n (FIG. 14) of the flat drain section is such as will snugly fit within the height and width g, g′ (FIG. 20) of the first rounded rectangular recess 142 in the rearward end 140 of the connector 130.

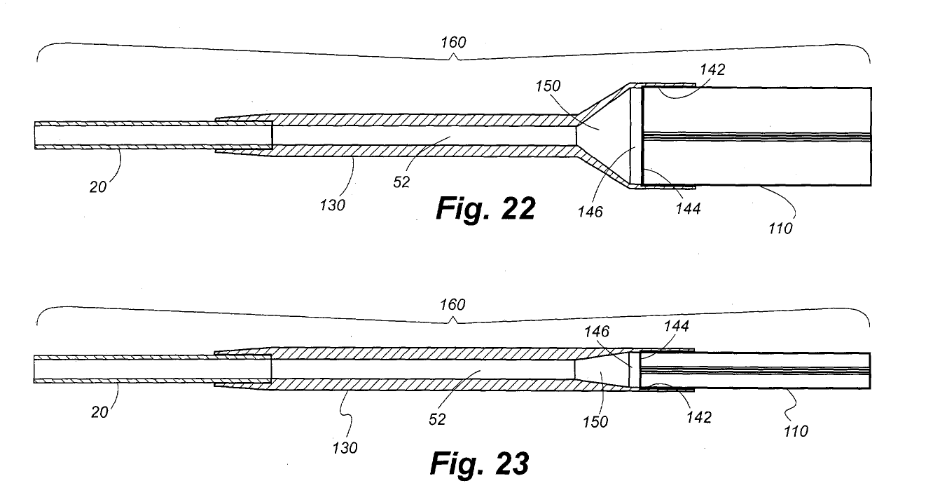

FIGS. 22 and 23 show a wound drain 160 comprising the suction tubing 20, flat drain section 110, and connector 130. The suction tubing 20 engages the forward portion 32 of the connector 130 in the same manner hereinabove described with respect to FIG. 11. At the rear of the connector 130, the flat drain section 110 has advanced until it confronts the base 144 of the rounded rectangular cavity 142, which acts as a stop. As can be seen in the figures, all of the longitudinal lumens of the flat drain section 110 empty into a common space within the connector 130, that being the rounded rectangular cavity 146 and taper 150 that leads into the cylindrical bore 52.

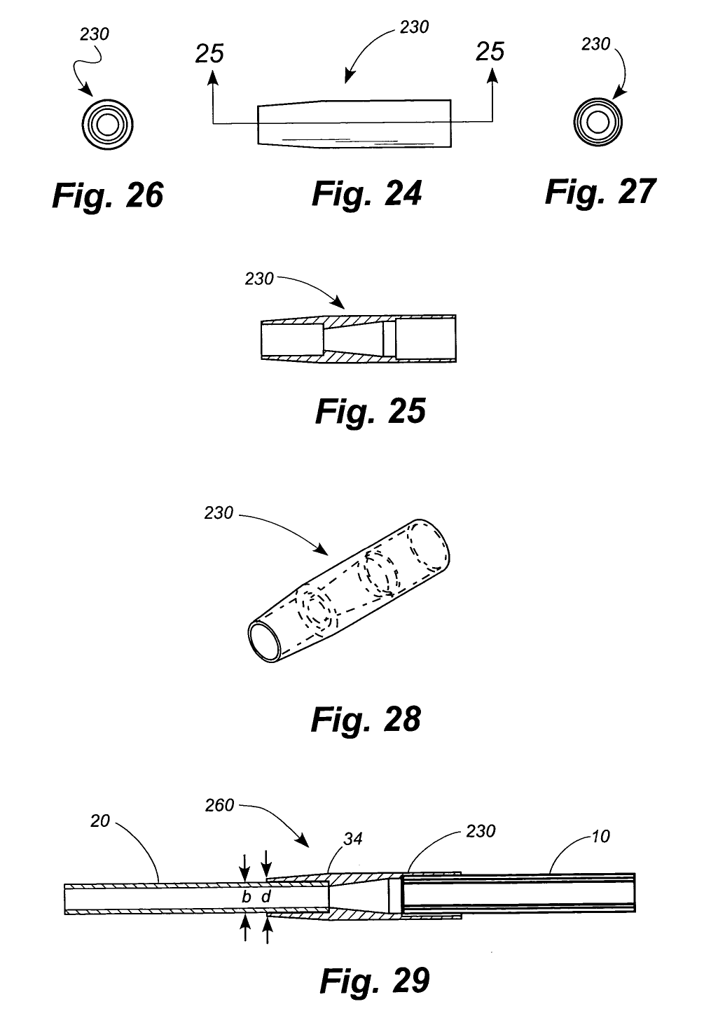

FIGS. 24-36 illustrate two embodiments of connectors for use in fully fluted wound drains. The connector 230 of FIGS. 24-28 is functionally identical to the connector 30 hereinabove described, except that the bore 52 which extends a major portion of the length of the connector 30 has been eliminated, along with its associated length, thereby dramatically shortening the length of the connector 230. Otherwise, the connector 230 interacts with a round drain section 10 and a suction tubing 20 in the same manner as hereinabove described with respect to the connector 30.

FIG. 29 illustrates a fully-fluted wound drain 260 comprising a round drain section 10, a suction tubing 20, and a connector 230. With a fully-fluted wound drain, because of the short length of the connector, it is conventional to implant the entire connector 230 within the body of the patient, leaving only the major portion of the suction tubing 20 exposed outside the patient's body. With the connector 230 and the transition between the connector and the suction tubing embedded within the tissues of the patient, any steps or edges on the connector or between the suction tubing and the connector can tear at tissue when the wound drain is moved. Consequently, the connector 230 is designed to eliminate step ups and edges that can tear at tissue. For example, the outer diameter b of the suction tubing 20 is 0.193 inches, and the outer diameter d of the end of the connector 230 is 0.199 inches, a difference of only 0.006 inches. From the end of the connector to the shoulder 34, a distance of 0.313 inches, the diameter tapers smoothly from 0.199 to 0.302 inches. The profile along one side of the tube changes according to the difference in radius. Thus in this case the radius changes by 0.056 inches over a distance of 0.313 inches, which calculates to a slope of 0.16, again with no steps or edges that can tear at tissue. Thus when the time comes to remove the wound drain 260 from the patient, the physician grasps the suction tubing 20 and exerts a tension. The connector 230, because of the absence of edges, gently pushes or spreads the tissue apart instead of tearing or ripping it. The smaller drain tubing 10 follows along in the hole made by the connector 230. There is less resistance applied to the circumference of the drain tubing because it is being pulled through a hole greater than its diameter. Thus the damage to the tissue done by removal of the wound drain 260 is minimal.

While the connector 230 of the present invention has a forward end having an outer diameter d that is only 0.006 inches greater than the diameter of the suction tubing 20, a difference of as much as 0.026 inches or less can give satisfactory results, with improved results being achieved with a difference of 0.016 inches or less, and preferred results being achieved at 0.006 inches or less.

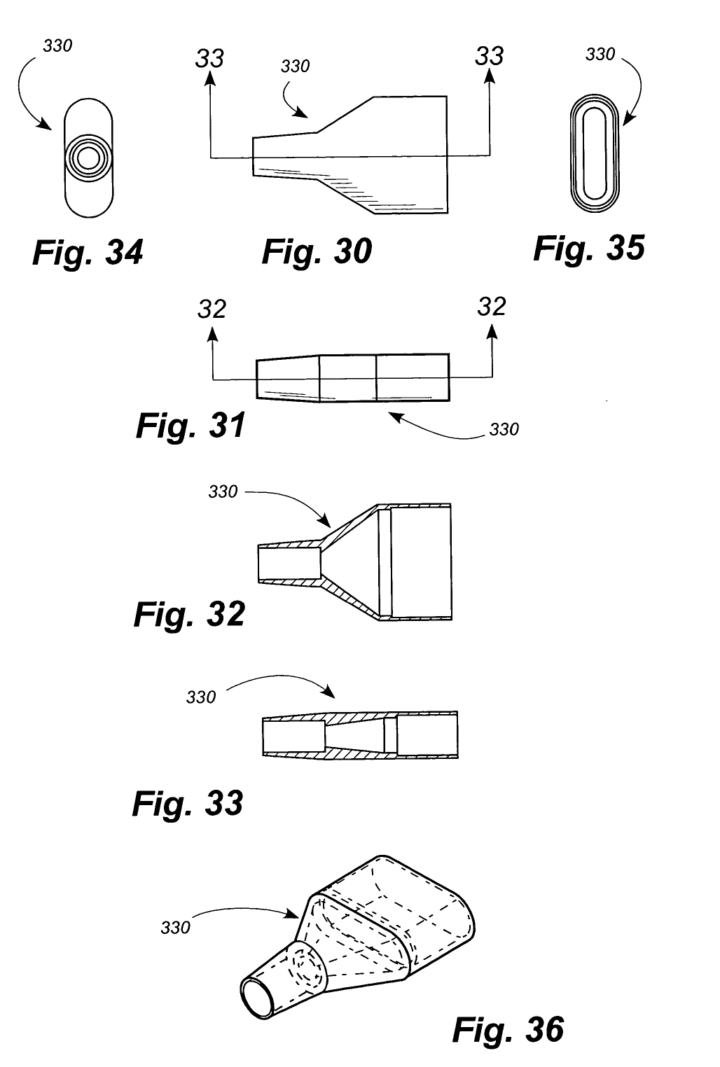

The connector 330 of FIGS. 30-36 is functionally identical to the connector 130 hereinabove described, except that the bore 52 which extends a major portion of the length of the connector 130 has been eliminated, along with its associated length, thereby dramatically shortening the dimensions of the connector 330. Otherwise, the connector 330 interacts with a flat drain section 110 and a suction tubing 20 in the same manner as herein above described with respect to the connector 130.

In the case of fully fluted wound drains employing the connectors 230, 330 for a wound drain of approximately one foot in length, the connector is only approximately one inch or less in length, meaning the associated drain section is slightly over eleven inches in length. Thus well over 90% of the length of the wound drain is fluted. A consequence of this arrangement is that the fully fluted design is best suited for wounds that are on the surface or less than an inch beneath the surface, as the connectors 230, 330 are incapable of traversing healthy, nonexuding tissue thicker than this length. In such an instance, either the drain section or the suction tubing will be left to traverse the healthy tissue. In the case of the drain section traversing healthy tissue, chances are high of the drain section becoming clogged with tissue, or of healthy tissue becoming injured. In the case of the suction tubing traversing healthy tissue, chances are high of the tubing, which has a relatively low resistance to radial forces, becoming partially crimped off. Once the walls are displaced closely together, the suction can draw the walls completely together, closing off the suction lumen and disabling the wound drain.

While the drain sections of the foregoing embodiments have been disclosed with respect to examples employing longitudinal slots for placing the lumens in fluid communication with the ambient surrounding the drain sections, it will be understood that other means, such as discontinuous grooves, holes, and the like may be used as alternatives to the uninterrupted slots. Further, while the foregoing embodiments have utilized interior walls to divide the interior into four lumens, it will be understood that a greater or lesser number may be used. Finally, while none of the longitudinal lumens are in fluid communication with one another, and all of the lumens are in direct fluid communication with the suction tubing, it will be appreciated that arrangements are possible in which some longitudinal lumens are in fluid communication with one another, and some lumens are only indirectly in communication with a vacuum source such as by way of other longitudinal lumens.

Of the embodiments described above, the three-quarter fluted wound drain has a connector 30, 130 that is approximately three inches in length, while the full fluted wound drain has a connector 230, 330 that is less than approximately one inch in length. It is possible that partially fluted designs of less than three-quarters fluted may be contemplated, or that specialty designs that are more than three-quarters but less than fully fluted may be contemplated. For purposes of this application, a connector that is greater than approximately two inches in length and having a lumen 52 of at least one inch in length will be considered to be doing more than merely interconnecting the drain portion and the suction tubing of the wound drain, i.e., it will be considered to be intentionally lengthening the wound drain, such as for the purpose of providing an intermediate section of enhanced radial strength.

Finally, it will be understood that the disclosed embodiments have been disclosed by way of example, and that other modifications may occur to those skilled in the art without departing from the scope and spirit of the appended claims.

What is claimed is:

1. A wound drain, comprising:
an elongated drain portion, said drain portion comprising an exterior wall defining a hollow interior, said drain portion comprising walls dividing said interior into a plurality of longitudinal lumens, and said exterior wall defining means for placing said longitudinal lumens in communication with the ambient surrounding said drain portion;
a suction tubing having a lumen capable of being placed in fluid communication with a source of suction; and
a connector for connecting a first end of said drain portion to a first end of said suction tubing such that said longitudinal lumens are placed in fluid communication with said lumen of said suction tubing,
whereby said ambient surrounding said drain portion is capable of being placed in fluid communication with a source of suction by means of said wound drain; and
wherein said connector exhibits increased radial strength in comparison to said drain portion and said suction tubing; and
wherein said connector is at least two inches in length.

2. The wound drain of claim 1, wherein said drain portion has a substantially round cross section.

3. The wound drain of claim 1, wherein said drain portion has a substantially rounded rectangular cross section.

4. The wound drain of claim 1, wherein said exterior wall defining means for placing said longitudinal lumens in communication with the ambient surrounding said drain portion comprises said exterior wall defining a plurality of longitudinal slots therethrough, at least one slot associated with each of said longitudinal lumens for placing said longitudinal lumens in fluid communication with the ambient surrounding said drain portion.

5. The wound drain of claim 1, wherein said exterior wall defining means for placing said longitudinal lumens in communication with the ambient surrounding said drain portion comprises said exterior wall defining a plurality of ports therethrough, at least one port associated with each of said longitudinal lumens for placing said longitudinal lumens in fluid communication with the ambient surrounding said drain portion.

6. The wound drain of claim 1, wherein said connector is approximately one-third the length of said elongated drain portion.

7. A wound drain, comprising:
an elongated drain portion, said drain portion comprising an exterior wall defining a hollow interior, said drain portion comprising walls dividing said interior into a plurality of longitudinal lumens, and said exterior wall defining means for placing said longitudinal lumens in communication with the ambient surrounding said drain portion;
a suction tubing having a lumen capable of being placed in fluid communication with a source of suction; and
a connector for connecting a first end of said drain portion to a first end of said suction tubing such that said longitudinal lumens are placed in fluid communication with said lumen of said suction tubing, said connector maintaining said first end of said drain portion and said first end of said suction tubing in spaced apart relation and placing them in fluid communication by means of a lumen that is at least one inch in length;
whereby said ambient surrounding said drain portion is capable of being placed in fluid communication with a source of suction by means of said wound drain; and
wherein said connector exhibits increased radial strength in comparison to said drain portion and said suction tubing.

8. The wound drain of claim 7, wherein said drain portion has a substantially round cross section.

9. The wound drain of claim 7, wherein said drain portion has a substantially rounded rectangular cross section.

10. The wound drain of claim 7, wherein said exterior wall defining means for placing said longitudinal lumens in communication with the ambient surrounding said drain portion comprises said exterior wall defining a plurality of longitudinal slots therethrough, at least one slot associated with each of said longitudinal lumens for placing said longitudinal lumens in fluid communication with the ambient surrounding said drain portion.

11. The wound drain of claim 7, wherein said exterior wall defining means for placing said longitudinal lumens in communication with the ambient surrounding said drain portion comprises said exterior wall defining a plurality of ports therethrough, at least one port associated with each of said longitudinal lumens for placing said longitudinal lumens in fluid communication with the ambient surrounding said drain portion.

12. The wound drain of claim 7, wherein said connector is approximately one-third the length of said elongated drain portion.

13. A wound drain, comprising:
a connector having forward and rearward ends;
a suction tubing having a forward end comprising a forward end of said wound drain and having a rearward end connected to said forward end of said connector; and
an elongated drain section having a forward end connected to said rearward end of said connector;
wherein no portion of said wound drain, beginning from said forward end of said wound drain, has a diameter 0.026 inches less than an immediately rearwardly adjacent portion of said wound drain.

14. The wound drain of claim 13, wherein no portion of said wound drain, beginning from said forward end of said wound drain, has a diameter 0.016 inches less than an immediately rearwardly adjacent portion of said wound drain.

15. The wound drain of claim 14, wherein no portion of said wound drain, beginning from said forward end of said wound drain, has a diameter 0.006 inches less than an immediately rearwardly adjacent portion of said wound drain.
